(19) United States
(12) Patent Application Publication
(10) Pub. No.: US 2023/0190681 A1
(43) Pub. Date: Jun. 22, 2023

(54) Topical Ointment Composition, Methods Of Use, And Methods Of Preparation

(71) Applicant: Hector Gonzales, Brownsville, TX (US)

(72) Inventor: Hector Gonzales, Brownsville, TX (US)

(21) Appl. No.: 17/553,211

(22) Filed: Dec. 16, 2021

(51) Int. Cl. A61K 31/125; A61K 9/00; A61K 9/06; A61K 36/61; A61K 31/045; A61K 36/8962; A61K 33/00; A61K 47/10; A61K 47/06; A61K 36/185; A61K 36/15

(52) U.S. Cl. CPC A61K 31/125; A61K 9/0014; A61K 9/0043; A61K 9/06; A61K 36/61; A61K 31/045; A61K 36/8962; A61K 33/00; A61K 47/10; A61K 47/06; A61K 36/185; A61K 36/15

(57) ABSTRACT

A topical ointment composition for nasal and sinus decongestion includes camphor, eucalyptus oil, menthol, garlic paste, sodium bicarbonate, isopropyl alcohol, water, and petrolatum. The topical ointment composition is applied to nasal vestibules of a user to reduce nasal and sinus congestion.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ointment compositions and more particularly pertains to a new ointment composition for nasal and sinus decongestion. The present invention discloses an ointment composition comprising menthol, camphor, eucalyptus oil, and garlic in a petrolatum base.

### (2) Description of Related Art including information disclosed under 37 CFR 1.97 and 1.98.

The prior art relates to ointment compositions. Prior art ointment compositions for application to nasal vestibules may comprise combinations of hydroxypropylmethylcellulose and garlic, ointments comprising less than 1% total by weight of menthol, camphor, eucalyptus oil, and turpentine oil, as well as barrier forming compositions, which may comprise eucalyptus oil. What is lacking in the prior art is an ointment composition comprising at least 2.9% by weight of menthol, camphor, eucalyptus oil, and further including garlic.

### BRIEF SUMMARY OF THE INVENTION

An embodiment of the disclosure meets the needs presented above by generally comprising camphor, eucalyptus oil, menthol, garlic paste, sodium bicarbonate, isopropyl alcohol, water, and petrolatum. The topical ointment composition is configured for application to nasal vestibules of a user to reduce nasal and sinus congestion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a flow diagram for a method of preparing a topical ointment composition according to an embodiment of the disclosure.

FIG. 2 is a flow diagram for a method of using an embodiment of the disclosure.

### DETAILED DESCRIPTION OF THE INVENTION

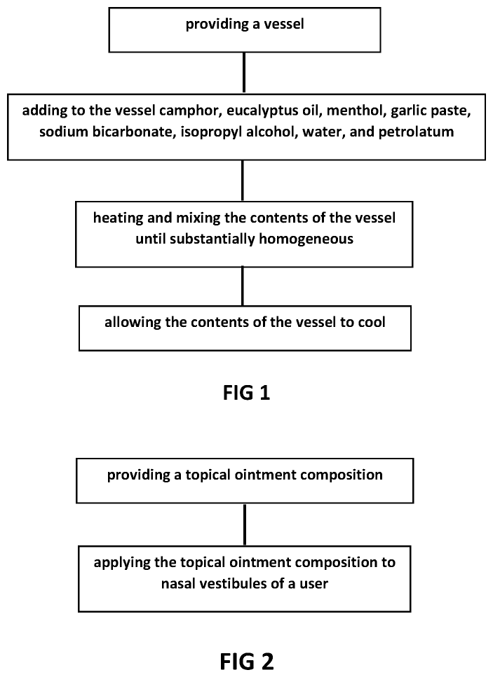

With reference now to the drawings, and in particular to FIGS. 1 and 2 thereof, a new ointment composition embodying the principles and concepts of an embodiment of the disclosure and generally designated by the reference numeral 10 will be described.

As best illustrated in FIGS. 1 and 2, the topical ointment composition 10 generally comprises camphor, eucalyptus oil, menthol, garlic paste, sodium bicarbonate, isopropyl alcohol, water, and petrolatum. The topical ointment composition is configured for application to nasal vestibules of a user to reduce nasal and sinus congestion.

The composition comprises 1.50 to 5.00 wt % camphor, 0.40 to 1.50 wt % eucalyptus oil, 1.00 to 4.00 wt % menthol, 10.00 to 25.00 wt % garlic paste, 5.00-20.00 wt % sodium bicarbonate, 11.00 to 12.00 wt % isopropyl alcohol, 0.00 to 7.50 wt % water, and 22.00 to 72.00 wt % petrolatum. The composition may comprise 2.40 to 2.80 wt % camphor, 0.06 to 0.07 wt % eucalyptus oil, 1.20 to 2.00 wt % menthol, 15.00 to 18.00 wt % garlic paste, 10.00-14.00 wt % sodium bicarbonate, 10.00 to 15.00 wt % isopropyl alcohol, 4.00 to 6.00 wt % water, and 42.13 to 57.34 wt % petrolatum.

The composition also may comprise one or more of turpentine oil, menthol, nutmeg oil, thymol, and cedarleaf oil. As such, the composition may comprise 1.50 to 5.00 wt % camphor, 0.40 to 1.50 wt % eucalyptus oil, 1.00 to 4.00 wt % menthol, 1.50 to 6.00 wt % turpentine oil, 1.50 to 6.00 wt % nutmeg oil, 0.04 to 0.12 wt % thymol, 1.00 to 3.00 wt % cedarleaf oil, 10.00 to 25.00 wt % garlic paste, 5.00-20.00 wt % sodium bicarbonate, 11.00 to 12.00 wt % isopropyl alcohol, 0.00 to 7.50 wt % water, and 9.88 to 67.06 wt % petrolatum. The composition may comprise 2.40 to 2.80 wt % camphor, 0.06 to 0.07 wt % eucalyptus oil, 1.20 to 2.00 wt % menthol, 2.25 to 3.00 wt % turpentine oil, 2.25 to 3.00 wt % nutmeg oil, 0.04 to 0.06 wt % thymol, 1.75 to 2.25 wt % cedarleaf oil, 15.00 to 18.00 wt % garlic paste, 10.00-14.00 wt % sodium bicarbonate, 10.00 to 15.00 wt % isopropyl alcohol, 4.00 to 6.00 wt % water, and 33.82 to 51.05 wt % petrolatum.

The present invention includes a method of preparing a topical ointment composition for use in reducing nasal and sinus congestion. The method comprises a first step of providing a vessel. A second step of the method is adding to the vessel camphor, eucalyptus oil, menthol, garlic paste, sodium bicarbonate, isopropyl alcohol, water, and petrolatum. A third step of the method is heating and mixing the contents of the vessel until substantially homogeneous. A fourth step of the method is allowing the contents of the vessel to cool so that a topical ointment composition forms.

The topical ointment composition enables a method of reducing nasal and sinus congestion. The method comprises a first step of providing a topical ointment composition according to the specification above. A second step of the method is applying the topical ointment composition to nasal vestibules of a user. The method may comprise an additional one or two application per day of the topical ointment composition to the nasal vestibules of the user, thus up to three applications per day.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A topical ointment composition comprising camphor, eucalyptus oil, menthol, garlic paste, sodium bicarbonate, isopropyl alcohol, water, and petrolatum, wherein the topical ointment composition is configured for application to nasal vestibules of a user for reducing nasal and sinus congestion.

2. The topical ointment composition of claim 1, wherein the composition comprises 1.50 to 5.00 wt % camphor, 0.40 to 1.50 wt % eucalyptus oil, 1.00 to 4.00 wt % menthol, 10.00 to 25.00 wt % garlic paste, 5.00-20.00 wt % sodium bicarbonate, 11.00 to 12.00 wt % isopropyl alcohol, 0.00 to 7.50 wt % water, and 22.00 to 72.00 wt % petrolatum.

3. The topical ointment composition of claim 2, wherein the composition comprises 2.40 to 2.80 wt % camphor, 0.06 to 0.07 wt % eucalyptus oil, 1.20 to 2.00 wt % menthol, 15.00 to 18.00 wt % garlic paste, 10.00-14.00 wt % sodium bicarbonate, 10.00 to 15.00 wt % isopropyl alcohol, 4.00 to 6.00 wt % water, and 42.13 to 57.34 wt % petrolatum.

4. The topical ointment composition of claim 1, further including the composition comprises one or more of turpentine oil, menthol, nutmeg oil, thymol, and cedarleaf oil.

5. The topical ointment composition of claim 4, wherein the composition comprises 1.50 to 5.00 wt % camphor, 0.40 to 1.50 wt % eucalyptus oil, 1.00 to 4.00 wt % menthol, 1.50 to 6.00 wt % turpentine oil, 1.50 to 6.00 wt % nutmeg oil, 0.04 to 0.12 wt % thymol, 1.00 to 3.00 wt % cedarleaf oil, 10.00 to 25.00 wt % garlic paste, 5.00-20.00 wt % sodium bicarbonate, 11.00 to 12.00 wt % isopropyl alcohol, 0.00 to 7.50 wt % water, and 9.88 to 67.06 wt % petrolatum.

6. The topical ointment composition of claim 5, wherein the composition comprises 2.40 to 2.80 wt % camphor, 0.06 to 0.07 wt % eucalyptus oil, 1.20 to 2.00 wt % menthol, 2.25 to 3.00 wt % turpentine oil, 2.25 to 3.00 wt % nutmeg oil, 0.04 to 0.06 wt % thymol, 1.75 to 2.25 wt % cedarleaf oil, 15.00 to 18.00 wt % garlic paste, 10.00-14.00 wt % sodium bicarbonate, 10.00 to 15.00 wt % isopropyl alcohol, 4.00 to 6.00 wt % water, and 33.82 to 51.05 wt % petrolatum.

7. A method of preparing a topical ointment composition for use in reducing nasal and sinus congestion, the method comprising the steps of:
providing a vessel;
adding to the vessel camphor, eucalyptus oil, menthol, garlic paste, sodium bicarbonate, isopropyl alcohol, water, and petrolatum;
heating and mixing the contents of the vessel until substantially homogeneous; and
allowing the contents of the vessel to cool, such that a topical ointment composition forms.

8. The method of claim 7, wherein the topical ointment composition prepared by the method comprises 1.50 to 5.00 wt % camphor, 0.40 to 1.50 wt % eucalyptus oil, 1.00 to 4.00 wt % menthol, 10.00 to 25.00 wt % garlic paste, 5.00-20.00 wt % sodium bicarbonate, 11.00 to 12.00 wt % isopropyl alcohol, 0.00 to 7.50 wt % water, and 22.00 to 72.00 wt % petrolatum.

9. The method of claim 8, wherein the topical ointment composition prepared by the method comprises 2.40 to 2.80 wt % camphor, 0.06 to 0.07 wt % eucalyptus oil, 1.20 to 2.00 wt % menthol, 15.00 to 18.00 wt % garlic paste, 10.00-14.00 wt % sodium bicarbonate, 10.00 to 15.00 wt % isopropyl alcohol, 4.00 to 6.00 wt % water, and 42.13 to 57.34 wt % petrolatum.

10. The method of claim 7, wherein the topical ointment composition prepared by the method comprises one or more of turpentine oil, menthol, nutmeg oil, thymol, and cedarleaf oil.

11. The method of claim 10, wherein the topical ointment composition prepared by the method comprises 1.50 to 5.00 wt % camphor, 0.40 to 1.50 wt % eucalyptus oil, 1.00 to 4.00 wt % menthol, 1.50 to 6.00 wt % turpentine oil, 1.50 to 6.00 wt % nutmeg oil, 0.04 to 0.12 wt % thymol, 1.00 to 3.00 wt % cedarleaf oil, 10.00 to 25.00 wt % garlic paste, 5.00-20.00 wt % sodium bicarbonate, 11.00 to 12.00 wt % isopropyl alcohol, 0.00 to 7.50 wt % water, and 9.88 to 67.06 wt % petrolatum.

12. The topical ointment composition of claim 11, wherein the topical ointment composition comprises 2.40 to 2.80 wt % camphor, 0.06 to 0.07 wt % eucalyptus oil, 1.20 to 2.00 wt % menthol, 2.25 to 3.00 wt % turpentine oil, 2.25 to 3.00 wt % nutmeg oil, 0.04 to 0.06 wt % thymol, 1.75 to 2.25 wt % cedarleaf oil, 15.00 to 18.00 wt % garlic paste, 10.00-14.00 wt % sodium bicarbonate, 10.00 to 15.00 wt % isopropyl alcohol, 4.00 to 6.00 wt % water, and 33.82 to 51.05 wt % petrolatum.

13. A method of reducing nasal and sinus congestion utilizing a topical ointment composition, the method comprising the steps of:
providing a topical ointment composition comprising camphor, eucalyptus oil, menthol, garlic paste, sodium bicarbonate, isopropyl alcohol, water, and petrolatum; and
applying the topical ointment composition to nasal vestibules of a user;

14. The method of claim 14, wherein the topical ointment composition is applied an additional one or two times per day to the nasal vestibules of the user.

15. The method of claim 13, wherein the topical ointment composition comprises 1.50 to 5.00 wt % camphor, 0.40 to 1.50 wt % eucalyptus oil, 1.00 to 4.00 wt % menthol, 10.00 to 25.00 wt % garlic paste, 5.00-20.00 wt % sodium bicarbonate, 11.00 to 12.00 wt % isopropyl alcohol, 0.00 to 7.50 wt % water, and 22.00 to 72.00 wt % petrolatum.

16. The method of claim 15, wherein the topical ointment composition comprises 2.40 to 2.80 wt % camphor, 0.06 to 0.07 wt % eucalyptus oil, 1.20 to 2.00 wt % menthol, 15.00 to 18.00 wt % garlic paste, 10.00-14.00 wt % sodium bicarbonate, 10.00 to 15.00 wt % isopropyl alcohol, 4.00 to 6.00 wt % water, and 42.13 to 57.34 wt % petrolatum.

17. The method of claim 13, wherein the topical ointment composition comprises one or more of turpentine oil, menthol, nutmeg oil, thymol, and cedarleaf oil.

18. The method of claim 17, wherein the topical ointment composition comprises 1.50 to 5.00 wt % camphor, 0.40 to 1.50 wt % eucalyptus oil, 1.00 to 4.00 wt % menthol, 1.50 to 6.00 wt % turpentine oil, 1.50 to 6.00 wt % nutmeg oil, 0.04 to 0.12 wt % thymol, 1.00 to 3.00 wt % cedarleaf oil, 10.00 to 25.00 wt % garlic paste, 5.00-20.00 wt % sodium bicarbonate, 11.00 to 12.00 wt % isopropyl alcohol, 0.00 to 7.50 wt % water, and 9.88 to 67.06 wt % petrolatum.

19. The method of claim 18, wherein the topical ointment composition comprises 2.40 to 2.80 wt % camphor, 0.06 to 0.07 wt % eucalyptus oil, 1.20 to 2.00 wt % menthol, 2.25 to 3.00 wt % turpentine oil, 2.25 to 3.00 wt % nutmeg oil, 0.04 to 0.06 wt % thymol, 1.75 to 2.25 wt % cedarleaf oil, 15.00 to 18.00 wt % garlic paste, 10.00-14.00 wt % sodium bicarbonate, 10.00 to 15.00 wt % isopropyl alcohol, 4.00 to 6.00 wt % water, and 33.82 to 51.05 wt % petrolatum.
